(19) United States
(12) Patent Application Publication
(10) Pub. No.: US 2004/0153160 A1
(43) Pub. Date: Aug. 5, 2004

(54) Implant for vertebral replacement and restoration of the normal spinal curvature

(76) Inventor: Mauricio Rodolfo Carrasco, Federal Capital (AR)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/696,774

(22) Filed: Oct. 30, 2003

(30) Foreign Application Priority Data
Oct. 30, 2002 (AR) P02 01 04138

(51) Int. Cl. A61F 2/44

(52) U.S. Cl. 623/17.15

(57) ABSTRACT

An implant for vertebral replacement comprised by inner supports to the vertebral plates, vertebral separating means and vertical members for adjusting the implant to the corresponding vertebral faces, wherein the piece of the inner support implant emulates the shape of the cortical tissue area of the vertebral plate thereby forming a frame and leaving the spongy tissue area of the vertebral plate free.

This invention in general relates to methods of fixing and replacing vertebrae and spinal arthrodesis, and particularly it refers to an implant for the replacement for vertebral bodies, their respective intervertebral disks and a method of insertion into and fixation in the spinal column. Particularly, this invention relates to a vertebral implant for the surgical prosthetic replacement of vertebrae and method of column reconstruction. Said implant is composed of flat frame shaped or trapezoidal, triangulate or rectangular ring-shaped members, emulating the perimeter area of vertebral plates and a telescopic motion column system which may be adapted in its length according to the column defect to be replaced, side supports to be fixed in the vertebral wall and to be constructed as a wedge, slanted bars of tubes to restore the spinal curvature in the replaced area. Furthermore, the implant devices have determined functions and may be exchanged to adjust heights, adjustability, adaptation to the type of vertebra (sacral, lumbar, thoracic or cervical) and the spinal angle to be restored, thus enabling the use of the most convenient implant set once the surgical area has been opened.

### BACKGROUND

Vertebral injuries which restoration is essential arise from a plurality of causes such as cancerous injuries, fractures caused by vertebral traumatism or vertebral softening such as osteoporosis and vertebral deformity of degenerative origin. Said injuries may harm the normal structure of vertebral bodies and, as a consequence, they may cause column deformities, pain and instability of the supporting structure of the skeleton thus compromising the nervous system, the medulla and its nerves, and causing pain and disabilities and even possible permanent damage.

Among the proposed treatments, the surgical treatment is specifically possible which aims to the neurological damage repair by decompressing the compressed nervous tissue and generating a mechanically stable vertebral segment. In order to achieve said goal, it is necessary that the skeletal defect be replaced and stabilized by means of various osteosynthesis methods to provide for a temporary stability and with the addition of bone grafts, biologically active substances or surgical cement, a longer duration of the altered column segment repair is obtained.

In order to achieve a mechanically efficient bone union, it is necessary to meet several biological and osteosynthesis requirments:

a) use of bone grafts and/or biologically active materials, which must be placed abundantly due to the normal volume loss occurring during the bone callus formation process, and if said goal is not achieved, the structure that has been made may be mechanically weak and thereafter it may be destroyed upon the body weight load and its motions,

b) the most possible contact surface between the vertebral bone ends and the bone grafts in order to favor the formation of the bone callus, since the lack of contact or the contact carried out in small surfaces does not form callus. mechanically strong enough to support loads,

c) preferably, those healthy vertebral ends with which said grafts are contacted must have the best blood irrigation in order to favor the rapid incorporation of grafts, and, consequently, it is preferable that said grafts contact the most central part of the vertebra where the so called cancellous bone is with a significant blood irrigation,

d) the osteosynthesis must be fixed in and supported by mechanically resistant bone tissues, said support depending on the stability and durability of the mounts interposed in the spinal column, since it is a structure that must support considerable axial loads and therefore the best areas of the vertebral surfaces for said support are the peripheral areas structurally being the continuation of the vertebral cortical walls,

e) supporting surface of implants with a minimum surface to prevent them from sinking in the bone and causing their subsequent instability in the mount or loss of correction of the spinal column axis upon the collapse of the construction made,

f) said implants must neutralize mechanical loads in the three spatial plans in an equivalent way, otherwise they should associate with various mount systems complementing them,

g) said implants must restore the natural curvature of the spinal column,

h) implants for vertebral replacement, characterized in that they are placed by means of a previous surgical intervention, require the best visual field of the spinal medulla to avoid projecting the graft over the medulla thereby damaging it.

In order to achieve said goals, various implants and their methods of use have been proposed, specifically by means of the intervention of the spinal column in its anterior part and removal of the damaged vertebrae and intervertebral disks. Among the implants known for this purpose, some of them may be mentioned which features are not appropriate for the above-mentioned goals and some examples thereof are provided for hereinbelow.

Implants that are not appropriate to the concept of the above item (a) are disclosed in U.S. Pat. No. 5,236,460 by Barber, which relates to a solid device with solid supporting plates which does not enable to arrange the bone in a significant way between the vertebrae; U.S. Pat. No. 6,190,201 by Sutcliffe, U.S. Pat. No. 6,193,755 B1 by Metz-Stavenhagen and U.S. Pat. No. 5,571,192 by Schonhoffer, refer to closed implants that do not enable the adaptation of the graft to the volume or to the necessary closeness to the vertebral ends since said implants must be filled with grafts before being placed in the spinal column defect to be repaired, thus causing the loss of the appropriate contact between the grafts and vertebrae for it is unattached within the implant and without possibilities of arranging it after its implantation since the access to the closed cavity is through small holes.

Other implants that are not appropriate to the concept of the above-stated item (b) are disclosed in U.S. Pat. No. 4,932,975 by Main and U.S. Pat. No. 5,458,641 by Ramirez Jimenez, both of which have solid or porous platforms in their supporting ends with vertebrae, thus interfering with the close contact of the grafts with the vertebral bone.

Other implants that do not comply with the concepts established in items (c) and (d) are disclosed in U.S. Pat. No. 5,336,223 by Rogers U.S. Pat. No. 4,657,550 by Daher, U.S. Pat. No. 4,554,914 by Kapp, U.S. Pat. No. 4,553,273 by Wu, which implant ends rest on the core of the vertebrae, which is the area having the biggest mechanical weakness of the vertebra and the most optimum area for fixation of grafts for bone fixation, but said implants cannot be used for the same reason as previously stated.

Other implants that do not meet the requirements established in the above-mentioned item (e) are disclosed in U.S. Pat. No. 5,702,455 , by Saggar and U.S. Pat. No. 5,989,290 by Harms, both of which support vertebrae by means of laminar walls, thereby enabling the easy sinking and subsequent loosening and the possibility of causing the implant migration.

Furthermore, other implants that do not meet the mechanical requirements of the above-mentioned item “f” are disclosed in U.S. Pat. No. 5,443,515 by Averill, U.S. Pat. No. 5,290,312 by Kojimoto and Yasui, U.S. Pat. No. 5,571,190 by Ulrich and Wolf, U.S. Pat. No. 6,176,881 B1 by Schar, Hatebur and Schapfer, which implants only support axial loads and must be placed together with other osteosynthesis systems, such as plates with screws that neutralize those forces to which the vertebral segment is subjected, in other space plans. In pursuit of this goal, U.S. Pat. No. 5,916,267 by Tienboon discloses two leaves in the implant ends which are attached at a right angle to the main body of the implant and with a lateral extension to the vertebrae to be fixed with screws, but since said extension is fixed, it does not enable the adaptation to the relative and variable angles of the vertebral bodies in their normal curvature configuration. Likewise, U.S. Pat. No. 5,290,312 by Kojimoto and Natsuo and U.S. Pat. No. 6,159,211 by Boriani et al do not enable said adaptation to the different vertebral angles in the different spinal column levels. There have been other attempts to stabilize the construction in the three spatial plans, such as the addition of further fixation means to the lateral faces of the vertebrae added to the main element placed between said vertebrae. Said characteristics are exemplified in U.S. Pat. No. 5,236,460 by Barber, wherein said fixation means are fixed to the platform of each end, without having the possibility of adaptation to the changes of the vertebral angles, since its extension is at a right angle both in the outlet of the implant body and in its extension and it cannot vary according to the vertebral anatomical changes. In U.S. Pat. No. 6,106,557 by Robioneck et al, a lateral plate is added to the main body of the implant. Said plate is fixed in the vertebrae by means of screws, which is similar to one of the variants proposed in U.S. Pat. Nos. 5,702,453 and 5,776,198 by Rabbe, wherein a lateral plate is added to the end of the main body of the implant with the same constructive criterion as the above-mentioned patent, since they disclose a variant referring to an extension coming from the platforms of both ends wherein a bar is articulated and wherein said bar ends in a plate having holes for its adaptation to the lateral faces of the vertebrae and its screwing. Another variant of lateral extensions to be fixed to the lateral part of vertebral bodies is disclosed in U.S. Pat. No. 6,190,413 B1 by Sutcliffe, which has a “L” shaped arm to be screwed to the outer part of the main cylindrical body interposed between the vertebrae and by means of a lateral groove in said arm, where it rests on the vertebra, fixation screws are placed.

Another device is disclosed in U.S. Pat. No. 4,289,123 by H. K. Dunn, wherein the vertebrae are separated by means of two parallel bars, which may be adjusted with nuts to fix said separation and said bars are supported by side plates with corresponding holes to accept said bars, fixing said plates to the vertebral walls. Another variant of said device is described in U.S. Pat. No. 6,106,527 by Wu and Chen, wherein said bars are not free as the ones in Dunn's Patent but each of them originate in the corresponding side plates and fixation is achieved by means of screws which exert a perpendicular pressure on the bars and it further includes a central plate which lead the bars, reduce the flexing possibility of the bars and is attached to the bars with screws which exert a perpendicular pressure thereon. Both devices having only side plates do not reach the balance of mechanical loads thus forcing the structure of vertebral walls and causing as a consequence a mechanical instability. Furthermore, said devices do not teach any means for the restoration of the spinal curvature.

A usual methodology in the application of vertebral replacements and, specially, in the cervical area, consists in placing a cervical plate in order to fix it to the spinal column with screws. In this way, after having-been placed on the solid bone graft, the cervical plate fixes said graft and the vertebrae of the defect ends. This method, which is used in many occasions has several disadvantages, one of which consists in the fact that once the solid bone graft has been placed, the medulla cannot be seen and afterwards when handling the osteosynthesis plate, the graft may be projected into the medulla thereby damaging it without noticing it since it is hidden from view. Among other disadvantages, there is the fix arrangement of the holes in the plates, which turns their adaptation difficult to the places recommended for placing the screws in the vertebral bodies.

Other known systems different from the mentioned traditional osteosynthesis plates have the same difficulty since the require firstly the graft placement and then the immobilization system to be placed on said graft, such as the implants disclosed in U.S. Pat. No. 5,620,443 by Gertzbein et al and U.S. Pat. No. 6,193,720 B1 by Yuan et al. In said patents, a system of bars outside the spinal column enable the fixation of a graft previously placed between the vertebrae is disclosed.

There have been other attempts to repair the spinal column defect which resort to the placement of closed cages filled in with bone grafts, such as U.S. Pat. No. 6,231,610 B1 and Document WO 02/03885 A2 by Michelson. Both of them are useful for being placed between the neighbor vertebrae but they are not appropriate to supplement the lack of several vertebral segments.

Other examples of known implants useful for the replacement for several segments are the above-mentioned U.S. Pat. No. 6,159,211 by Boraini, and U.S. Pat. No. 5,192,327 by Brantingan. Both of them relate to implants consisting in closed cages, which, upon their placement in the required position and owing to the fact that they lack their own fixation means, need to be supplemented by other osteosynthesis means to keep the construction stability and cannot adapt themselves to the spinal column curvatures.

One of the known ways of obtaining an immediate fixation of mounts with the use of implants for vertebral replacement is the use of surgical cement instead of bone grafts. Said cement is generally used in the fixation of prosthesis to bones. Its more frequent use is for example the fixation of prosthesis for hips, knees and other minor joints. Its use has shown the need of a careful and systematic handling owing to essentially two characteristics of the material such as its exothermal reaction and its appropriate plasticity point. The exothermal reaction is the own characteristic of this kind of plastic material and it is triggered upon joining the liquid portion of the component with the acrylic powder. Said temperature is highly harmful for the nervous tissue, which must be protected. Therefore, the broad visual field of said nervous tissue and a space big enough to handle the acrylic cement is critical in order to avoid irreversible damages.

With regard to the plasticity point of the acrylic mass, it is obtained some minutes after its components have been bonded. The aspect of the appropriate mass to be handled and placed has a consistency similar to that of mastic so that the modeling made by the surgeon's hands enables a modeling appropriate for the cavity to be filled in or as long as necessary to join the vertebral bodies by penetrating its core in cavities previously made. In this way, the spilling of acrylic is avoided thereby preventing it from leaking into other sites where damage may be caused, such as nerves, arteries, or other tissues. Therefore, it is not recommended to use liquid acrylic cement, which in fact is the only form it may be used in hollow, tubular and/or closed implants, where there are small holes through which it may be injected. It is not safe to use it with implants that do not enable a broad visual field of the medulla and nerves, such as implants occupying the central part between the vertebrae and partially hiding the medulla with the risk of failing to notice some cement leakage to its surroundings.

The analysis and study of the prior art enables us make a quite correct classification of the different implants known in the state in the art.

We may group and name as closed system those implants that do not enable a clear visual field of the medulla and nerves and/or do not enable to handle fusion materials such as acrylic cement in the intervertebral area. Then, we may name as open system those implants that do in fact enable them.

Secondly, we may group and name as outer systems those implants that convey mechanical efforts through the outer vertebral faces. Then, we name as inner systems those systems that convey mechanical efforts through the inner area of the vertebral plate.

Examples of closed and inner implant systems are U.S. Pat. Nos. 4,932,975; 5,236,460; 5,290,312; 5,571,192; 5,702,453; 6,106,557; 6,159,211; 5,916,267; 5,360,430; 5,458,641; 6,395,030; 5,192,327; 5,360,430.

Examples of closed and outer systems are U.S. Pat. Nos. 6,193,720 and 6,306,136.

Examples of open and outer systems are U.S. Pat. Nos. 4,289,123; 6,106,527; 6,136,002; and 5,620,443.

Examples of open and inner systems are U.S. Pat. No. 5,062,850 and this invention.

With regard to the implant disclosed in U.S. Pat. No. 5,062,850, it must be pointed out that it lacks the basic properties to meet the requirements stated at the beginning of the background discussion. Said implant is composed of three fix bars and two solid outer plates, which do not enable the fusion between the bone material and the central spongy area of the vertebral plate.

Although the main characteristics of some known implants have been described as a reference, said characteristics not being appropriate for the pursued goal, they share in some aspects said peculiarities. Therefore, there is still a need of an implant for adapting it to mechanical and biological needs of vertebral fixation that facilitate the reconstruction of vertebral defects as well as its mechanical fixation, and the mechanical characteristics that said implant and its mount should have to improve the deficiencies of other implants are the following:

a) having a vertebral supporting base appropriate to maximize stability in the operation of mechanical efforts and enough to avoid any sinking in vertebrae, maximizing the contact area of the fusion material with the spongy tissue of the vertebral plate,

b) providing enough space for placing a considerable volume of bone grafts, or substitutes thereof, and for handling surgical cement or equivalent materials,

c) enabling the direct visual field of the nervous elements during the system mounting to avoid damages caused by implant or instrumental elements;

d) holding vertebrae and stabilizing in the three spatial plans with the own means of the implant;

e) enabling the natural restoration of the spinal curvature in the affected zone,

f) providing enough surface on the vertebral plates to favor the fusion of the bone grafts and the equivalent material with the vertebral body,

g) enabling determination of the approximate separation in situ between the opposite faces of the implant,

h) enabling the exact fixation in situ of the separation of the opposite faces of the implant.

Therefore, the object of this invention is an implant for vertebral replacement, which characteristics improve the deficiencies of other implants and enable a better use of the bone grafts for the definite stabilization of the spinal column.

### SUMMARY OF THE INVENTION

This invention relates to an implant for the vertebral body replacement and its use technique for repairing a defect in the spinal column. It provides for the immediate stabilization to definitely remain incorporated in the body and enables the use of bone grafts or other bioactive substances, or surgical cement, which contribute to the definite mount fixation.

Said implant in general comprises supporting devices on the cortical tissue of vertebral plates, a set of parallel columns composed of bars and tubes containing them thus forming a telescopic mechanism. Said bars are fixed to an end of the implant and the tubes, to the other end. Both ends are trapezoidal, triangulate or rectangular shaped platforms with extensions of shape of “E” facing letters, which configuration extends along the perimeter of the vertebral plate, and to which other accessory frames are fixed having the same shape but of different angles for the implant adaptation to the curvatures of the spinal column in the sagittal plan. The adaptation to the spinal column curvature is also achieved by leaning the bars and tubes with regard to the supporting devices. These accessory frames fix the implant to the vertebrae by means of screws in each of them. The length of said bars is predetermined as well as the length of the tubes, and they have different measures forming an exchangeable set. Said bars have also particular configurations which enable them to be cut at the required distance. By means of the selection of the appropriate set of said pieces, it is possible to form the total length of the implant to adapt it to the length of the spinal column defect to be repaired, as well as to restore the corresponding spinal curvature. Once the set of pieces appropriate to the case has been selected, the implant is placed in the spinal column defect and by means of the telescopic mechanism extension, the precise adaptation to the vertebrae of the defect ends of the spinal column is made. It is necessary to also have a predetermination system of the distance of the vertebral separation and incuts and discontinuities in order to provide an instrument and method for its placement. Finally, said telescopic mechanism is blocked by means of screws, and the fixation screws are placed on the vertebrae in both ends of the implant.

One of the objectives of this invention consists in providing for an implant with components having standard measures, adaptable in its length to the needs of each case, and with a robust construction for the spinal column stabilization in all the plans of physiological load of the spinal column.

Another objective of this invention consists in providing for an open implant, which enables the use of a considerable bone volume between the columns and since their ends are frames that leave a vertebral surface exposed in each end, said open implant enables an increased contact between vertebrae and grafts, thereby favoring the fixation thereof and the formation of a robust bone callus.

Another objective of this invention relates to the generation of a strong support for the axial load of the spinal column provided by a set of supporting columns arranged at the angles of a supporting frame in the periphery of the vertebral bodies, which is the structure having the best mechanical resistance, leaving the core of the vertebrae free, and being said core the most optimum part for bone fixation, to contact the bone graft mass.

Another objective of this implant consists in the mechanical stabilization in several spatial plans by means of fixation to the upper and bottom vertebral bodies by means of screws arranged in different spatial plans.

Among other benefits, during a surgical intervention, there is a benefit that consists in that the open form of the implant enables the permanent visual field of the medulla during all of the handlings of its mount and the placement of the grafts, thus avoiding any unnoticed damage of the nervous system.

### BRIEF DESCRIPTION OF THE DRAWINGS

FIG. A: General view of implant for the lumbar area including two bars

FIG. B.: View of the jig for angular correction

FIG. C.: View of bars at different lengths, and pieces with horizaontal slipping and inclined pieces.

FIG. D: General view of the implant for the cervical area including two bars

FIG. E: View of directions for screws and alternative cervical shape.

FIG. 1: Explosion view of implant I provided with appropriate components for the replacement for lumbar vertebrae or thoracic-lumbar vertebrae.

FIG. 2. Explosion view of implant II, provided with components for the replacement for lumbar vertebrae and the sacral bone.

FIG. 3: Oblique view of implant I placed between two lumbar vertebrae and the screws adjusting said vertebrae.

FIG. 4: Oblique view of implant II placed between a lumbar vertebra and the sacral bone.

FIG. 5: Side view of implant I placed between two lumbar vertebrae with vertebral fixation screws placed.

FIG. 6: Side view of implant II placed between one lumber vertebrae and the sacral bone, with vertebral fixation and telescopic system fixation screws

FIG. 7A: Front view of implant I placed between two lumbar vertebrae with vertebral fixation screws and telescopic system fixation screws.

FIG. 7B: View of piece 1, top view of the implant with a cut line AA.

FIG. 7C: View of pieces 1 and 2 assembled and placed under the lumbar vertebra, wherein the cut AA of piece 1 and the adaptation of the vertebra by a removal from the vertebral body are shown

FIG. 8: Front view of implant II placed between a lumbar vertebra and the sacral bone with its fixation screws, specially showing the ones that are fixed to the sacral bone.

FIG. 9A: Lateral view of piece 2 where the telescopic system tubes and a cut line are seen.

FIG. 9B: Lateral view of piece 1 by the cut line AA, where the direction of the threaded inner tubes for the placement of screws blocking the telescopic system.

FIG. 10A: General view of the assembled implant I and the approached vertebral supporting platforms, prepared to be placed in the vertebrae.

FIG. 10B: General view of the assembled implant I and with the vertebral supporting platforms separated between the vertebrae and the telescopic mechanism blocked by the corresponding screws.

FIG. 11A: Explosion view of implant III provided with appropriate components for the replacement between thoracic vertebrae.

FIG. 11B: Lateral view of an implant placed with vertical extensions for its lateral fixation.

FIG. 12: Scheme of a predetermination system between bars and indented tubes.

FIG. 13A: Top view of lumbar, thoracic and cervical vertebral plates, and indication of the cortical tissue.

FIG. 13B: Top view of lumbar, thoracic and cervical vertebral plates and indication of the shapes provided to the vertebral supporting pieces.

FIG. 14A: Explosion view of implant IV provided with appropriate components for the replacement between cervical vertebrae.

FIG. 15: Oblique view of a mounted cervical implant.

FIG. 16: Lateral view of an implant where the angular correction achieved by wedged pieces is seen.

FIG. 17: Oblique view of an implant supported by a nipper designed for the placement thereof.

FIG. 18: Lateral view of an implant held by the nippers designed for its placement and exhibited between two vertebrae.

FIG. 19A: Oblique rear view of an adaptation of the cervical implant for its placement in the axis bone.

FIG. 19B: Oblique anterior view of an adaptation of the cervical implant for its placement in the axis bone.

FIG. 19C: Oblique rear view of an adaptation of the cervical implant placed in the axis bone.

FIG. 20: Explosion view of a mount alternative system of an implant for vertebral replacement.

FIG. 21: Oblique view of an implant held by holding nippers.

FIG. 22: Oblique view of an implant with a system of stops for the fixation screws to the vertebral bodies.

### DETAILED DESCRIPTION OF THE PREFERRED EMBODIMENTS OF THE INVENTION

The following description includes several embodiments to carry out the invention and it aims to illustrate the general principles of its use and it must not be considered as limiting the possibilities thereof, its claims being more representative of its scope.

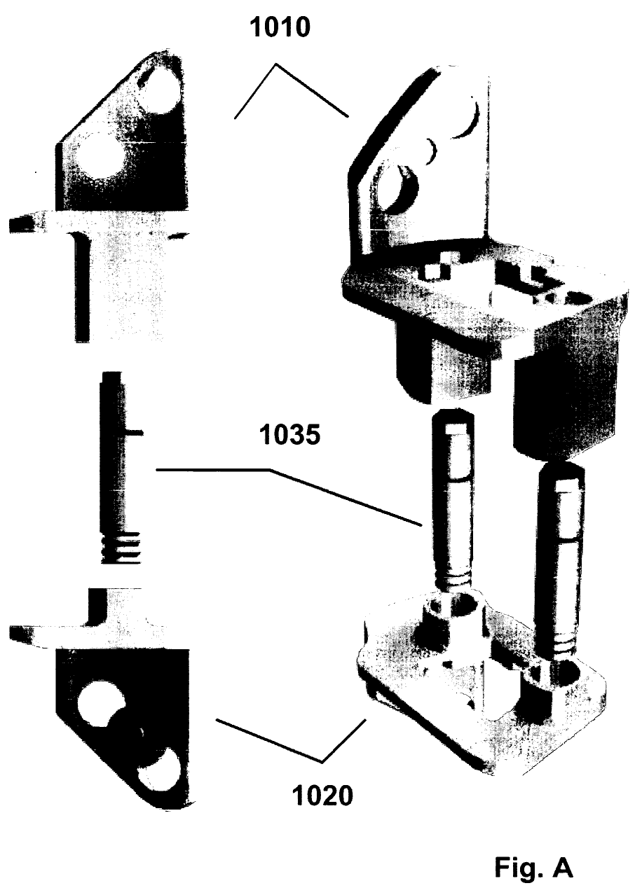

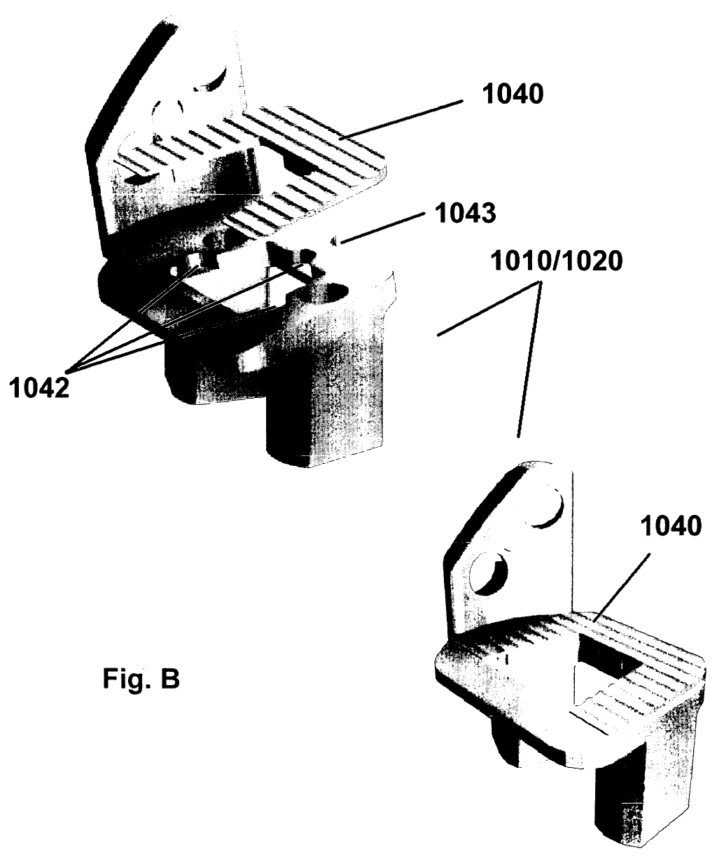

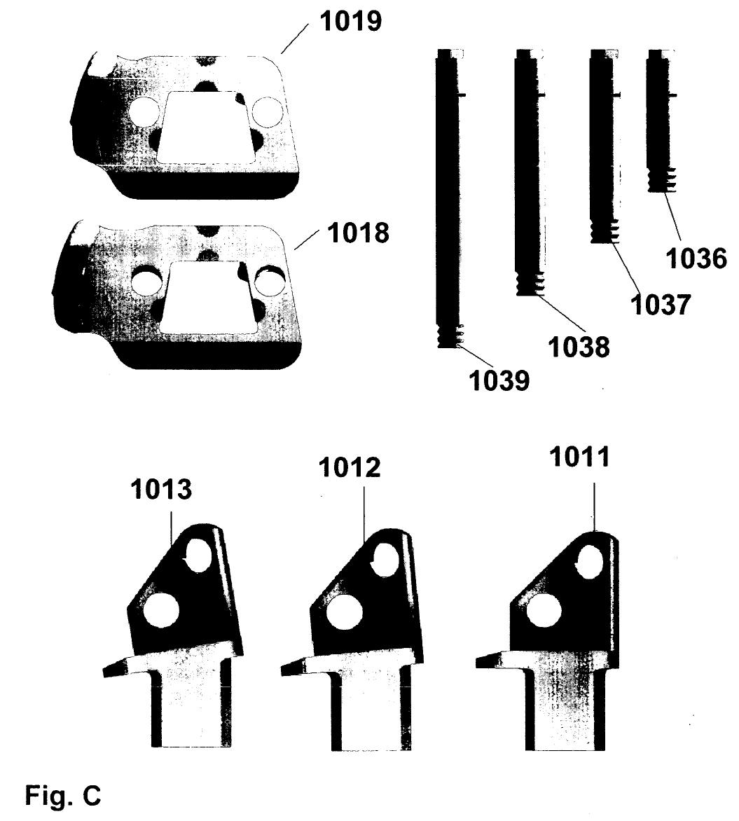

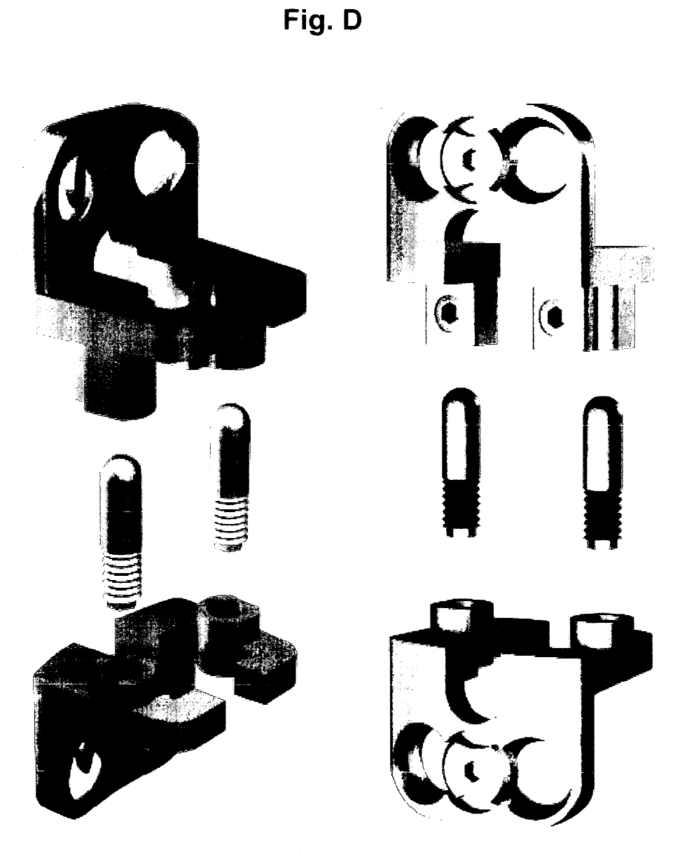

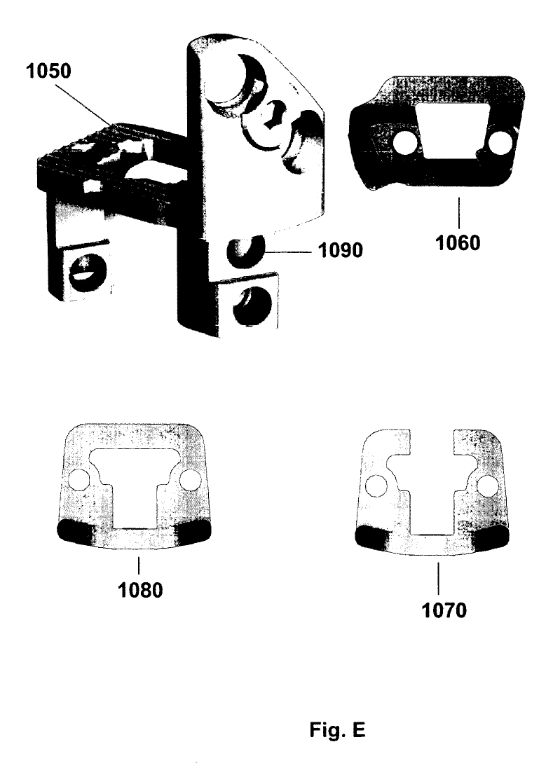

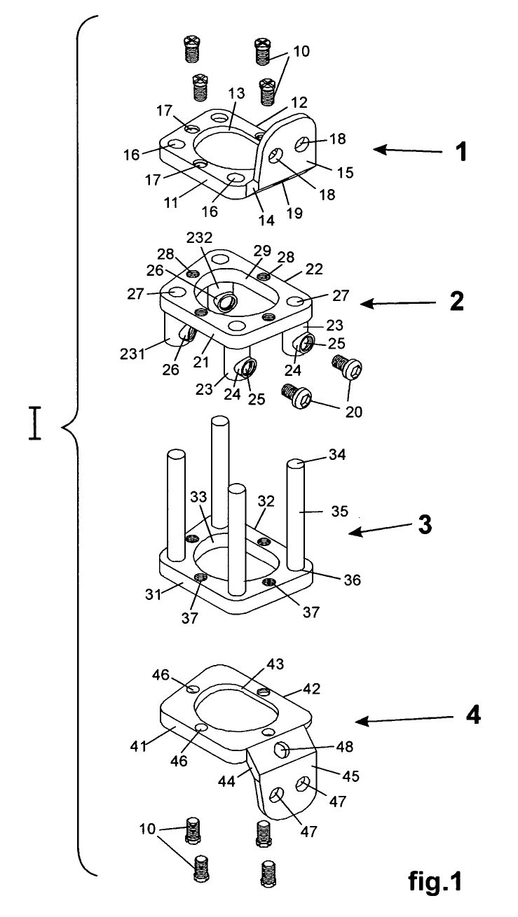

In a preferred embodiment, in FIG. 1, an explosion view of implant I is shown in an assembly embodiment to be placed between two lumbar vertebrae.

The parts thereof are the following:

Piece 1 is a top vertebral supporting frame composed of a trapezoidal shaped horizontal wedged member corresponding to the perimeter of the vertebral plate of the lumbar vertebrae and a vertical extension for the adjustment thereof to the lateral vertebral face.

Piece 2 is a telescopic adjusting frame, with a plan horizontal member having a trapezoidal shape suitable for its adjustment to piece 1 and four vertical extensions consisting in tubes perpendicular to the horizontal member which will receive the bars of piece 3.

Piece 3 is a vertebral separation frame, with a plan horizontal frame having a trapezoidal shape suitable for its adjustment to piece 4 and four vertical bars consisting in bars perpendicular to the horizontal member which will introduce themselves in the tubes of piece 4.

Piece 4 is a bottom vertebral supporting frame, composed of a trapezoidal shaped horizontal wedged member corresponding to the perimeter of the vertebral plate of the lumbar vertebrae and a vertical extension for its adjustment to the lateral vertebral face.

Piece 1 comprises a trapezoidal ring shaped flat member with an outer anterior and short side 11 and an outer rear opposite and longer side 12, parallel between each other, and an outer right side and a vertical left extension 14 that joins the anterior and rear non parallel sides thus closing the frame. The frame vertices are curved. Its hollow interior is determined by the interior perimeter 13.

The height of the section corresponding to the anterior side 11 is higher than the height of the section corresponding to the rear side 12, thus forming a kind of wedge that enables restoration of the preexisting angle between the vertebrae.

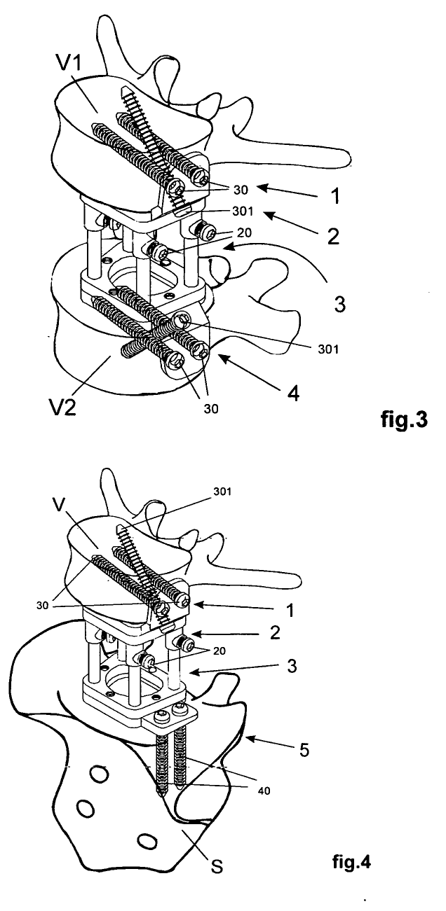

The lateral extension 14 has a hole 19 and continues on the laminar surface 15, which has two holes 18. Said two holes 18 and the hole 19 have a size for the passage of screws 30 and 301 shown in FIGS. 3, 4, 5 and 6. The arrangement of said holes enable the spatial fixation of the piece upon providing it with three fixation plans defined by the three screws 30 and 301. Piece 1 has four holes 17 perpendicular to their thickness for the passage of four screws 10, and four holes 16, each of them in each angle, having a size for the passage of bars 35.

Piece 2 comprises a trapezoidal ring shaped flat member with an outer anterior short side 21 and an outer rear opposite and longer side 22, both being parallel sides, and a right side and a left side that join at the anterior and rear side thus closing the frame which corresponds with piece 1. The vertices of the frame are curved. The hollow interior of the frame is determined by the inner perimeter 29.

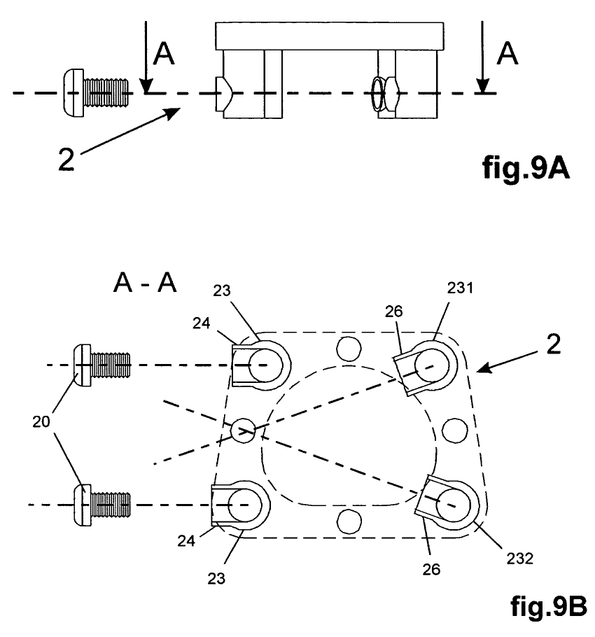

It has two attached tubes 231 and 232 and two tubes 23, all of them with attached tubes 26 and 24 with an inner thread 25 where four screws 20 are threaded. The attached tubes are parallel to each other and convergent 26 and oriented to a middle point between both tubes 23 to enable the introduction of a screwdriver between both tubes 23, to adjust the screws 20 in the tubes 26 as shown in FIG. 9B.

The frame 2 is adapted to frame 1 as indicated in the explosion view of FIG. 1, coinciding the edges 11 and 12 with the edges 21 and 22 respectively, and by means of screws 10 which penetrate into the smooth holes 17 and are threaded in the threaded holes 28, both pieces 1 and 2 are fixed and in this way the four holes 16 of frame 1 are in continuity with the holes of frame 2 for the passage of bars 35, upon introducing their ends 34 into the tubes 232, 232 and two tubes 23.

Piece 3 comprises a trapezoidal ring shaped flat member with an outer anterior short side 31 and an outer rear opposite and longer side 32, both being parallel sides, and a right side and a left side that join at the anterior and rear sides thus closing the frame which corresponds with piece 4. The vertices of the frame are curved. The hollow interior of the frame is determined by the inner perimeter 33.

Piece 3 includes four threaded holes for screws 10, and in each angle 36 a bar 35 is fixed with free ends 34.

Piece 4 comprises a trapezoidal ring shaped flat member with an outer anterior short side 41 and an outer rear opposite and longer side 42, both being parallel sides, and an outer right side and a vertical left extension 44 and 45 that join at the anterior and rear sides thus closing the frame. The vertices of the frame are curved. The hollow interior of the frame is determined by the inner perimeter 43

The height of the section corresponding to the anterior side 41 is higher than the height corresponding to the rear side 42, thus forming a kind of wedge that enables the restoration of the preexisting angle between the vertebrae.

The lateral extension 44 continues in extension 45. Said extension 44 has a hole 48, and extension 45 has two holes 47, said holes 48 and 47 being for the passage of screws 30 and 301, as may be seen in FIGS. 3, 4, 5 and 6 and they achieve the same spatial fixation as described for piece 1.

The assembly of pieces 3 and 4 is carried out as shown in FIG. 1, causing the anterior parts 31 and 41 to coincide with each other, and the rear parts 42 and 32 to coincide with each other. By means of screws 10 that penetrate through the holes 46, the threaded holes 37 are adjusted and pieces 3 and 4 are thereby formed.

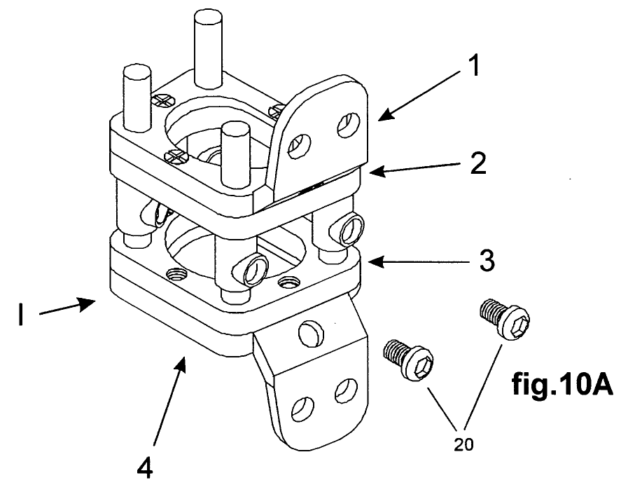

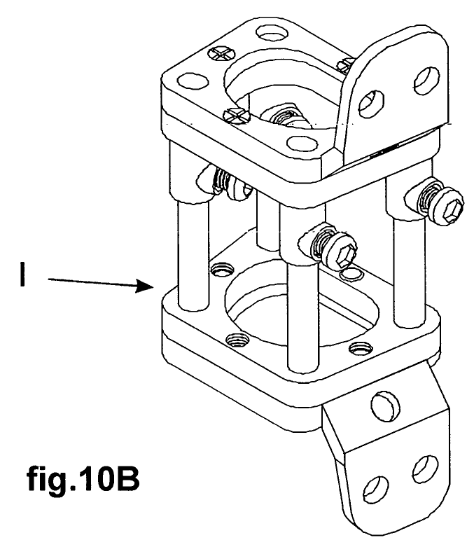

The assembly of the set of pieces of the upper end 1-2 with the set of pieces of the bottom end 3-4 is carried out as shown in FIG. 1, penetrating the bar ends 35 through the tube ends 231, 232 and the two tubes 23, so that the anterior parts 11, 21, 31 and 41 coincide with each other and the rear parts 11, 22, 32 and 42 also coincide with each other, thus forming a preferred embodiment of the formation of implant I, and comprising a telescopic motion device, enabling the approach or the separation of the ends of implant I as shown in FIGS. 10A and 10B respectively.

As an illustration of this preferred embodiment, FIG. 3 is an oblique image with implant I placed between two vertebrae V1 and V2; the assembled pieces 1, 2, 3 and 4 form implant I of FIG. 10B, and said implant is placed with the sutiable construction way for two lumbar vertebrae V1 and V2. Screws 30 and 301 are seen to fix vertebrae V1 and V2 and they are placed in ends 1 and 4. The screws 20 placed for blocking the telescopic system are also illustrated.

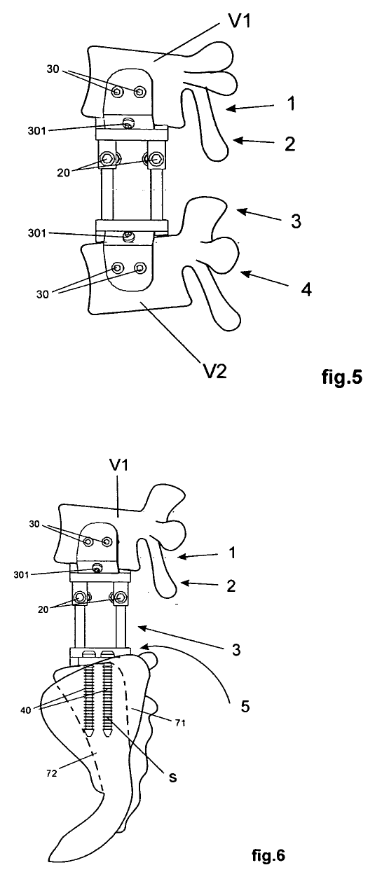

In FIG. 5, in a side view, implant I of FIG. 10B is shown with its constituent parts 1, 2, 3 and 4, placed between two lumbar vertebrae V1 and V2, with vertebral fixation screws 30 and 301, and with screws 20 for the telescopic system fixation.

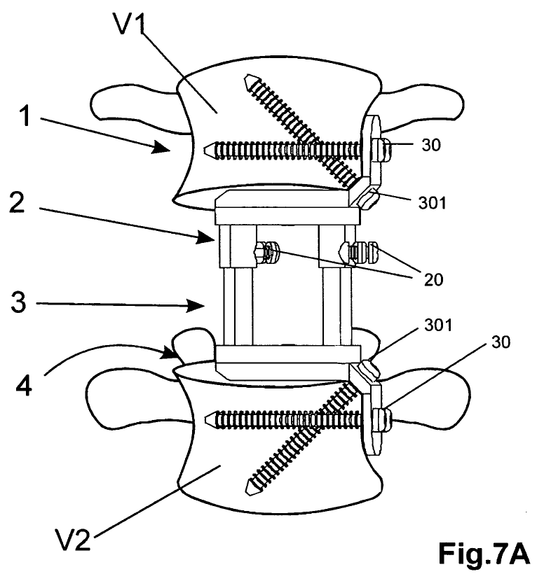

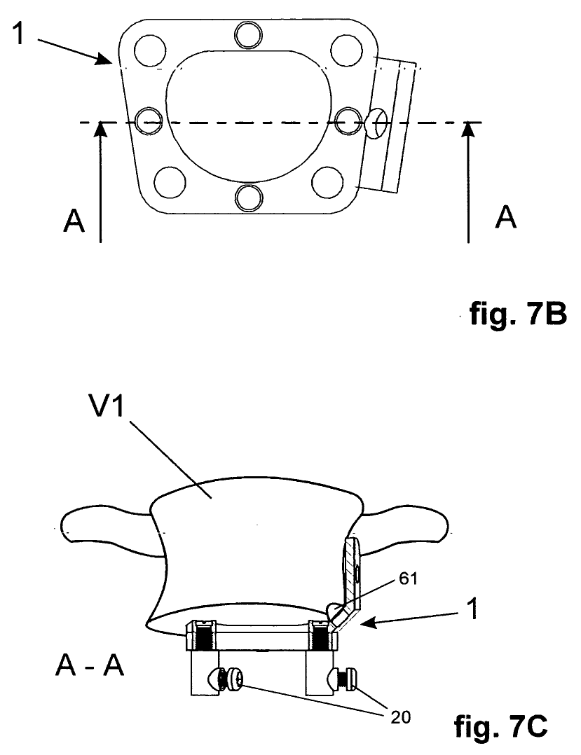

And in FIG. 7A, in a front view, an image of implant I is shown with its assembled parts 1, 2, 3 and 4, placed between two lumbar vertebrae V1 and V2. Screws 30 and 301 placed in vertebrae V1 and V2 and the screws 20 for the telescopic system fixation are also shown.

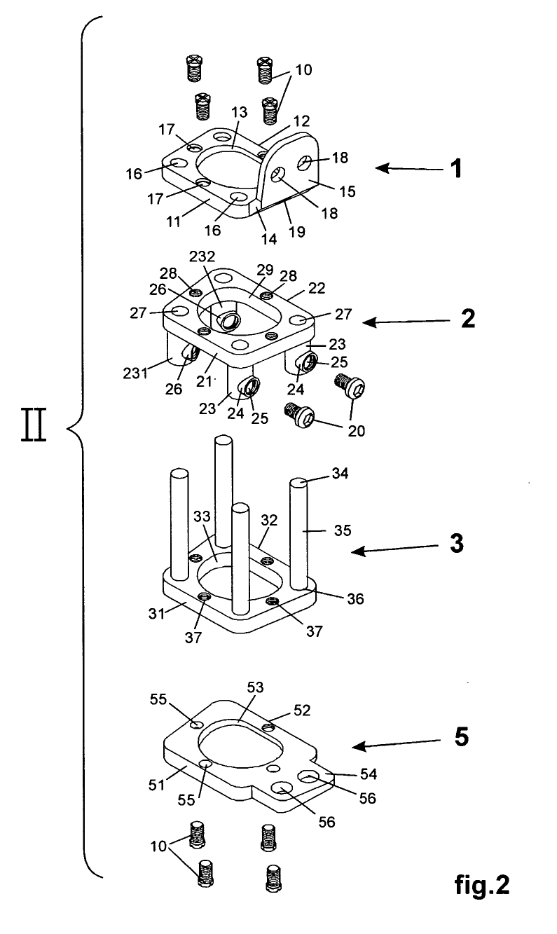

In another preferred embodiment, in FIG. 2, implant II is represented in a suitable way to be placed between a lumbar vertebra and the sacral bone. The component pieces 1, 2 and 3 of the implant have the same individual description as the above-provided description for the implant between lumbar vertebrae.

Piece 5 in FIG. 2, which is suitable for the sacral bone, has an anterior part 51 higher than the rear part 52, and a curved inner perimeter 53, which acquires a trapezoidal shape identical to frame 3, but with a lateral wedged aspect. There are four smooth holes 55 in its thickness for the passage of screws 10. In horizontal extension of its surface towards one of the sides 54, there are two holes 56 for the passage of screws 40, as seen in FIGS. 4, 6 and 8.

The assembly of implant II is carried out by firstly forming the set of pieces 1 and 2 as hereinabove described, and the set of pieces 3 and 5 causing its anterior parts 31 and 51 to coincide with each other and the rear parts 32 and 52 to also coincide with each other in order to afterwards fix both parts 3 and 5 by means of the screws 10 which penetrate through the holes 55 and are threaded in holes 37.

In another illustration of this preferred embodiment, in FIG. 4, in an oblique view, the assembly of implant of FIG. 1 is shown with its constituent parts 1, 2, 3 and 5, to be placed between a lumbar vertebra from the top, and the sacral bone from the bottom, where the set of pieces of the upper end 1-2 is assembled with the bottom end 3-5. In the bottom supporting piece, the arrangement of fixation screws 40 penetrating the sacral bone may be seen. Screws 30 and 301 are shown fixing vertebra V and screws 20 are shown blocking the telescopic system of the sacral-lumbar implant.

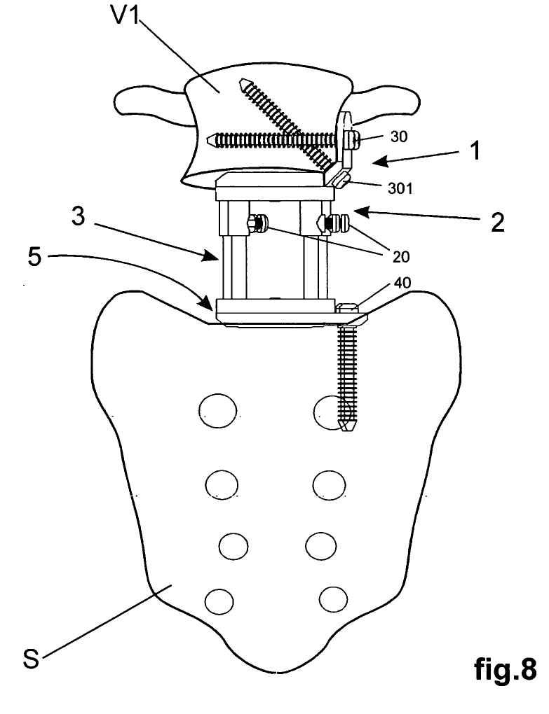

Another aspect of said mount is shown in FIG. 6, a side view of the scaral-lumbar implant of the assembly with its constituent parts 1, 2, 3 and 5, placed between the lumbar vertebra V1 and the sacral bone S. The lumbar vertebral fixation screws 30 and 301 are shown placed and screws 40 for fixation to the sacral bone are also shown. In FIG. 8, there is a front view of the assembly of implant II with its constituent parts 1, 2, 3 and 5, placed between a lumbar vertebra V1 and the sacral bone S. The lumbar vertebral fixation screws 30 and 301 and screws 40 for fixation to the sacral bone S may be seen. Likewise, the telescopic system fixation screws 20 may be seen.

A detail in the placement of the implant and its adaptation to the vertebrae of pieces 1 and 4 of the ends, is the need of removing a small portion of the vertebra 61 in its flange as seen in FIG. 7C for the suitable support of the lateral extension 14 and for the extension 44 with regard to the bottom vertebra.

Both of said preferred construction embodiments forming the sacral-lumbar and lumbar implants correspond to the need of preparing them to adapt the different vertebrae and in both ways, they comprise a telescopic system, which for constructive purposes, the load forces supporting the spinal column in a low level of the lumbar column were considered and for which the bars 35 had a diameter size of 4 mm, the frames of pieces 2 and 3 also have a minimum thickness of 4 mm, thus obtaining an enough strength of the structure when built with stainless steel or titanium alloys. Likewise, the additional ends 1, 4 and 5 of the implant add certain thickness to the bases of implants I and 11 and the pieces of said ends are joined by four screws 10, and without damaging the base strength, a system for the reciprocal insertion of the pieces may be added in its design thereby increasing its grabbing capability. The fixation of the formed telescopic system, the bars and the tubes are individually blocked to provide for a solid grabbing mechanism, with a support of the spinal column axial forces in four points, and a fixation to the vertebrae of at least two screws each.

Further to the sacral and lumbar area, it is also an object of this invention to provide the thoracic as well as the cervical area with the same replacement system, for which the corresponding adaptations are described hereinbelow.

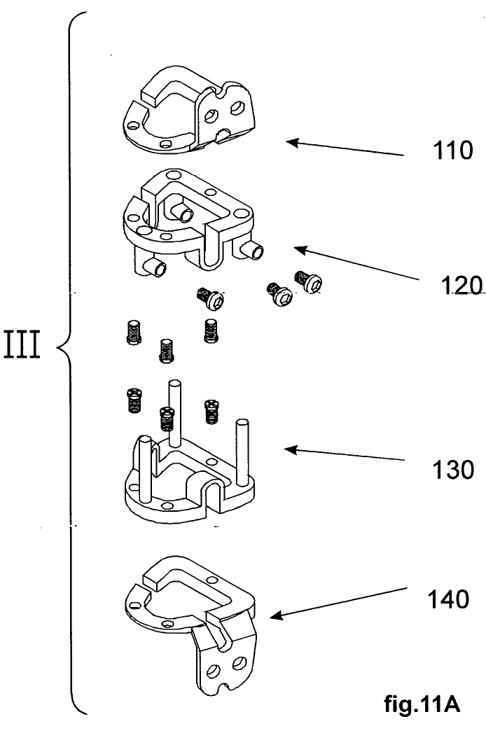

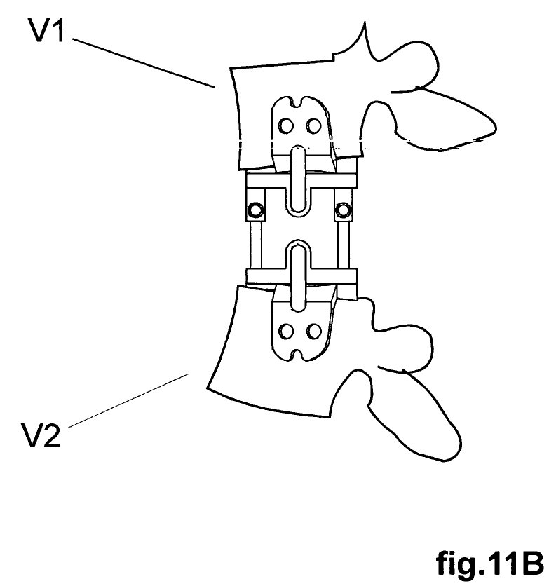

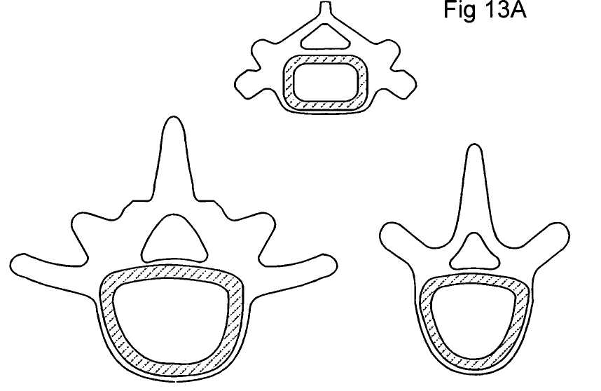

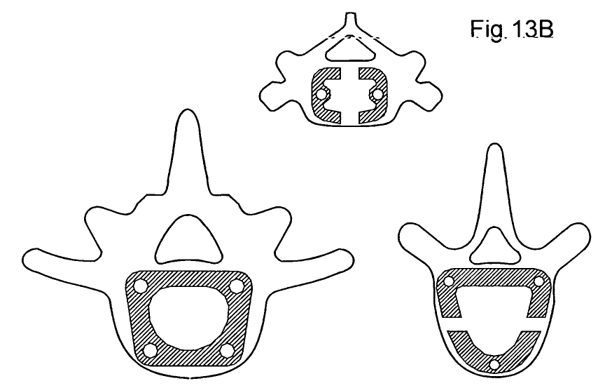

Particularly, and following the stability principle, the pieces comprising a vertebral replacement for the thoracic area are comprised with a triangulate shaped flat member as shown in FIGS. 11A, 13A and 13B, corresponding with the perimetrical shape of the thoracic vertebral plates and due to the triangulate configuration and in relation with the minor magnitude of the loads of said area and, as shown in FIG. 11A, 3 bars will be arranged in piece 130 with their corresponding 3 tubes in piece 120. Said bars and tubes will be located at the vertices of the triangulate configuration. In the same way, a threaded hole per side will be arranged for joining pieces 1 and 2, and 3 and 4. With regard to the wedge in pieces 110 and 140, it will have a side of a higher height and an opposite vertex of a lower height or vice versa.

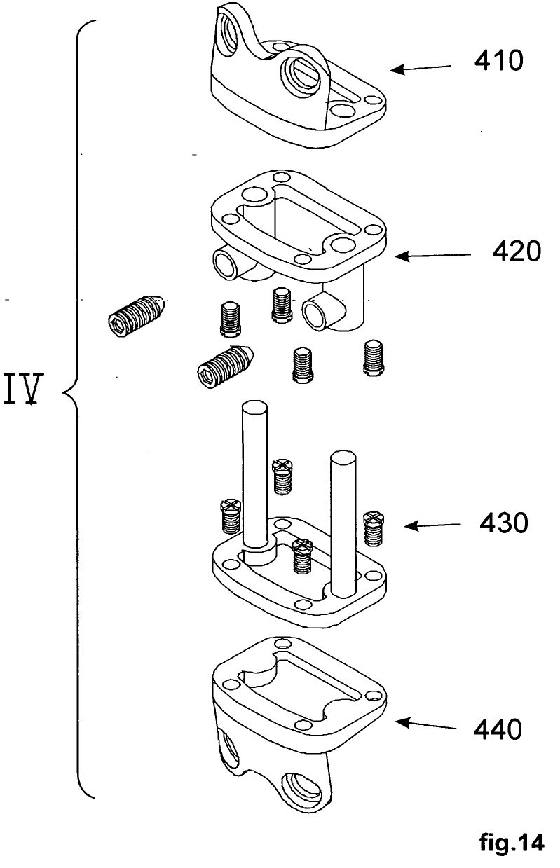

With regard to the pieces that form a vertical replacement for the cervical area, they will be comprised by a rectangular ring shaped flat member with an extension in the center of each smaller side as shown in FIGS. 14, 13A and 13B, which supports the bars and tubes, corresponding with the perimetrical form of the cervical vertebral plates, and owing to the rectangular configuration and in relation with the smaller load magnitude in said area, 2 bars will be arranged in piece 430 with their corresponding 2 tubes in piece 420. In the same way, a threaded hole will be provided for each bigger side for joining pieces 410 and 420 and pieces 430 and 440. The smaller sides with their extensions emulate an E shape.

The shapes given to the pieces according to the cortical surface in the vertebral plates are shown in FIGS. 13A and 13B.

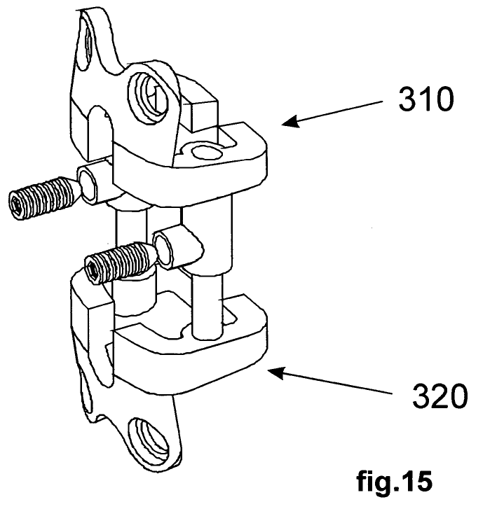

Particularly, for the replacement in the cervical area and owing to space requirements and the reduced mechanical loads, another preferred embodiment of this invention consists in using two pieces instead of four of them, wherein a piece A and a piece B are formed. Additionally, a series of incuts are indicated for a method of placement of the implant. Said mounted device is illustrated in FIG. 15.

Piece 310 is an upper vertebral supporting frame composed of a horizontal wedged member having a rectangular ring shape with an extension in the center of each smaller side serving as a support for the telescopic adjusting tubes, and a vertical extension having two central incuts, a bottom and a top incut, for using accessory instruments which may facilitate the preciseness in the placement thereof.

Piece 320 is a bottom vertebral supporting frame composed of a horizontal wedged member having a rectangular ring shape with an extension in the center of each smaller side serving as support for the vertebral separation bars, and a vertical extension having two central incuts, a bottom incut and a top incut, for using accessory instruments that facilitate preciseness in the placement thereof.

The horizontal member of the vertebral supporting frame, both top and bottom ones, may be interrupted in its continuation in the center of its opposite bigger side next to the place where the vertical extension originates, and said space is part of the incut set of the placement method by means of suitable instruments.

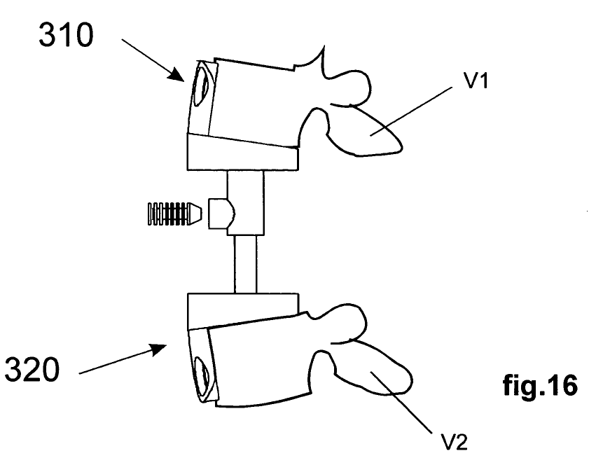

Both pieces have their corresponding holes for the insertion of screws, which will fix said pieces to the vertebral bodies as described in the previous embodiments. Likewise, the bars are blocked by means of the system described in the previous embodiments. The wedge effect on the horizontal members of the pieces are illustrated in the side view of an implant placed according to FIG. 16.

The two-piece system may also be suitable for thoracic or lumbar replacements, as long as material sufficiently resistant to the mechanical efforts to be supported is used. Under the same principle, the amount of bars used may be reduced.

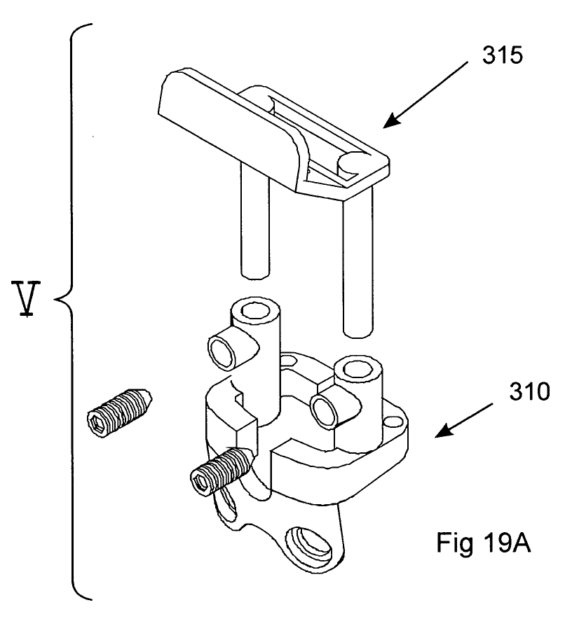

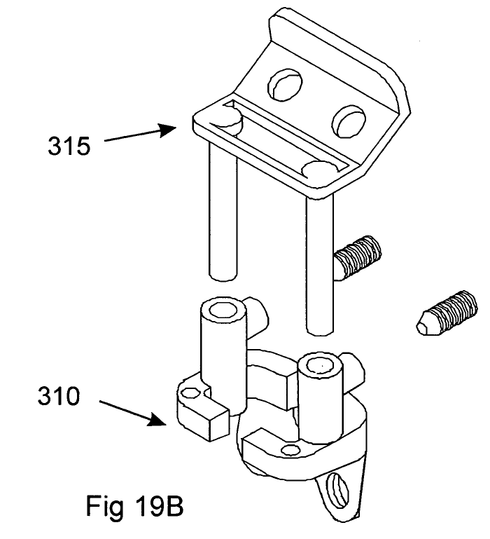

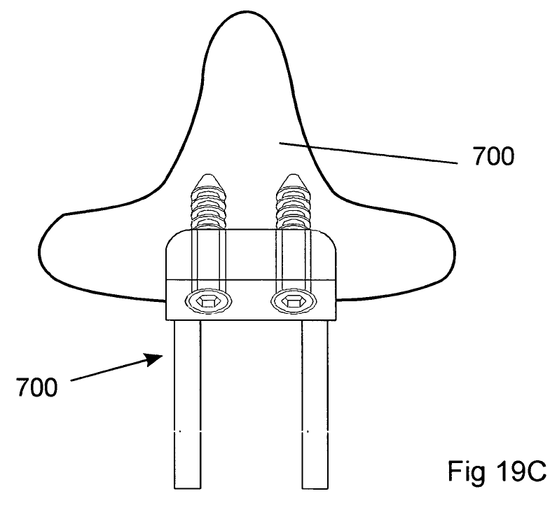

With regard to the cervical area, and in the same way as an adaptation becomes necessary for the bottom end of the spinal column to the sacral bone, the same happens with the axis bone. FIGS. 19A and 19B illustrate a variant composed of a piece 315 which may include bars or tubes as perpendicular projections, and which upper member consists of three flat members, one of them is horizontal and supports the bars, the second one is joined to the first one forming a 135 and 170° angle; and a third member following the previous one forming a 90° angle approximately. In the second member, there are two holes for the passage of screws to be introduced and which will adjust the axis bone, which owing to its structure cannot be correctly fixed at a vertical angle. FIG. 19C illustrates the adaptation placed in the axis bone. FIGS. 19A and 19B are examples of the inversion of bars and tubes, which may be indistinctly built in all the models with any of said configurations.

After having described the characteristics of each piece, the possibility of having variants of each one with predetermined angles, as well as predetermined tube and bar lengths becomes apparent in order to obtain, after surgery has been initiated and the deficiencies of the previous measurements have been experimented in situ, the implant set that adapts to the actual need of the particular case.

With regard to the angles obtained by means of wedged pieces, another preferred embodiment to achieve said angles consists in fixing the bars and tubes in a leaned way in relation with the flat members supporting them. For example, bars might be fixed at 8° with regard to the member supporting it and tubes at −8°, including any other required value, thus achieving the desired angular correction, and simplifying the production of supporting pieces. This angular correction system is more preferred for the two-piece implant. It is not convenient to bend the bars. They should be leaned instead since its curvature would be generating lateral efforts endangering the system stability.

With regard to vertebral separation bars, further to the possibility of having pieces of different bar lengths, another preferred embodiment of the invention comprises the alternative of shortening the bars by their ends in order to adapt their lengths. Said cuts usually originate small flanges which obstruct the free movement between the bars and tubes.

In order to overcome said problem, the hole composed of a tube and the hole in the piece supporting it and the hole in the supporting piece will form a hole with two different diameters: the diameter corresponding to the upper section (closest to the vertebral plate) will be slightly bigger than the diameter of the bottom section of the tube. In this way, the possible flanges will not obstruct the introduction of the bars into the tubes, and the process for the final adjustment of the separation between the opposite faces of the implant may be normally continued. This construction makes it possible that the set keeps the firmness for which it has been designed by keeping the diameter of the bottom section of the hole containing the bars adjusted to the diameter of the latter.

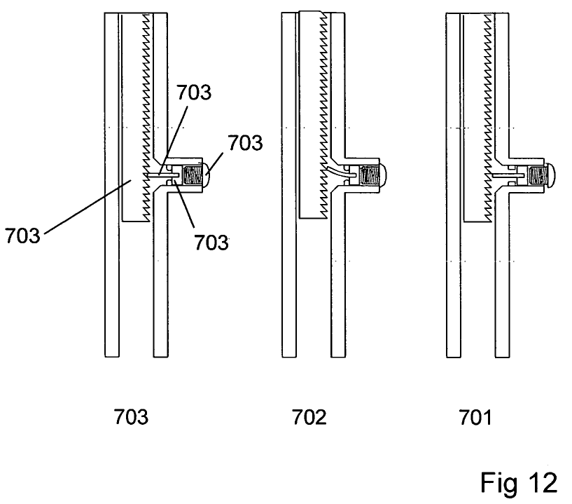

Another preferred embodiment to overcome the problem of flanges produced by the cut, consists in using diametrically grooved bars and said grooving may be either smooth or indented. The bar cut is carried out on the smallest diameter slits. In the case of an indented grooving, it has a second application since it may be used as a prefixation system of the implant set height. It is achieved by adding a semiflexible tooth or feather oriented towards the interior of the telescopic adjusting piece. FIG. 12 illustrates one of the various possibilities of this preadjusting system.

The obstruction device comprises an unidirectional flexible feather, fixed in the interior of the tube, at the height of the lateral hole in said tube, in its upper part, and is flexible towards the separation direction of the pieces. Among the different techniques used so that said feather is flexible in an only one direction, the use of a flexible curved metal sheet is described. Said curvature is obtained by fixing said metal sheet to the adjusting hole 25, in its upper section. In this way, the convex side efforts flex the feather thereby enabling the separating sliding of the pieces, and the concave side efforts resist said flexion by obstructing the bar at the desired height.

Said device will enable the sliding tending to separate the pieces and will obstuct the sliding tending to reduce the distance between the pieces. In this way, the surgeon, when placing the implant, may initiate the separation of the upper and lower pieces with the corresponding instruments, and once the desired separation has been reached, the surgeon may definitely adjust it with the screws without worrying about keeping the separation with his hands or by means of accessory instruments which may obstruct the operation field and make the adjustment handlings difficult.

Likewise, a tooth system may be used, which tooth is a triangulate with a rectangle, which horizontal side is perpendicular to the tube and is the bottom side of the triangle, and which vertical side is parallel to the tube. Of course, the tube includes an indentation of a triangulate rectangular shape where the horizontal side is the upper side and the oblique side is the bottom side. The upper side and the oblique side of the tooth, when pressure is exerted on the bottom and oblique sides of the indentation teeth of the tube, enable the sliding of the indented tube due to the flexion possibility of the sheet supporting the tooth and the space of the horizontal tube where the bar fixation screws will be afterwards placed. Once the desired height has been reached, the bars remain obstructed when contacting the two horizontal surfaces of the respective teeth. For a definite fixation of the height, the fixation screw 20 is placed in the threaded hole 25, which will exert pressure on the tooth against the bar, thereby obtaining an excellent fixation for the device.

Therefore, an object of this invention is an implant comprised by two pieces with an automatic height prefixing system, by means of prefixing means between the bars and the tubes, as well as an angular correction system consisting in fixing the bars and tubes leaning towards the flat member supporting them. Furthermore, the bars may be cut at the necessary height, in the diametrical grooves having a small diameter to be used for said purpose.

In order to free the system from the exclusive manual ability of the professional, the implant may have incuts and discontinuities in is frame as illustrated in FIG. 11A for thoracic implants and in FIG. 15 for cervical implants. Said configuration is not illustrated but it is extensible to the lumbar implant.

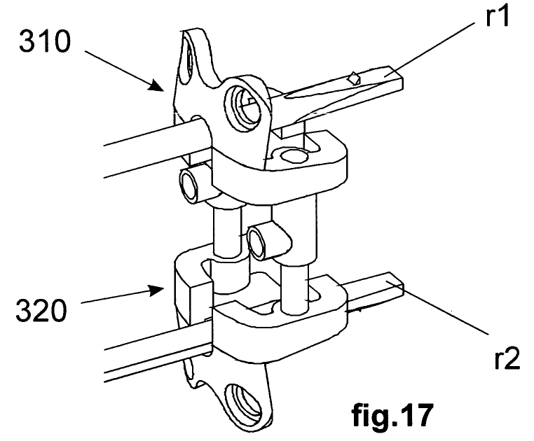

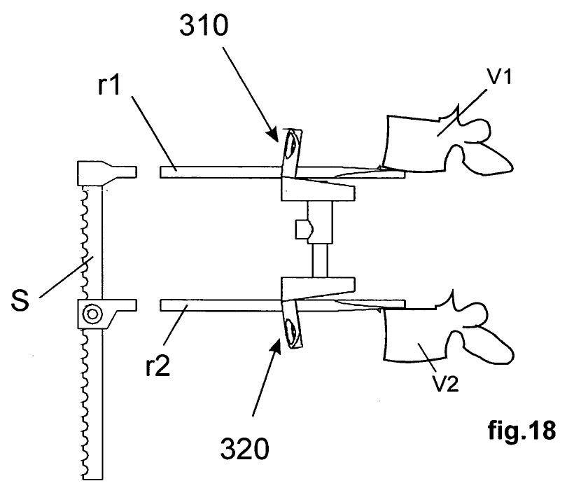

FIG. 17 illustrates the use of an instrument for the implant placement. The instrument consists in nippers or “scissors” that include stops near its ends R1 and R2. The upper and lower pieces supported with their incuts and they slid along the nippers arms up to the stops. Said stops contact the lateral wall of the vertebrae, as shown in FIG. 18. In this way, the implant is presented. The desired separation of both the implant and the vertebrae is obtained by exerting pressure with the nippers and then the implant is slipped until it is positioned in the intervertebral space. The nippers may be easily removed by reducing the pressure thereon and owing to the discontinuity of the horizontal frames and, in this way, the piece is ready for its final adjustment. The prefixing system enables the nippers removal without causing any collapse of the set. In this way, the surgeon will have a free operation field to make the definite adjustments by means of screws used for said purpose.

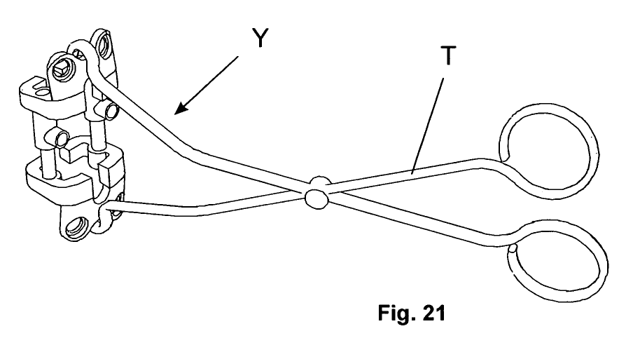

FIG. 21 illustrates alternative nippers for the placement of an implant by standard methods.

In order to illustrate with regard to one of the preferred embodiments for the implant placement of this invention, the following, non limiting, method is described:

1. By an intervention anterior to the column, the injured vertebral bodies with their respective disks are removed and the bone surfaces are prepared by removing its cartilage in its entirety and leaving the bone exposed.

2. Assembly of the four-piece implant: it is carried out outside the patient. Assembly of pieces 1 and 2: an accessory piece 1 is selected, which angle must be suitable for its adaptation to the upper vertebra V1, and piece 2 is put thereon causing the anterior parts 11 and 21 and rear parts 12 and 18 to coincide with each other. In this way, pieces 1 and 2 coincide as well as their four holes 16 with the four holes 27, on the one hand, and the four holes 17 coincide with the four holes 18, on the other. Screws 10 are placed in the holes 17, and they are screwed in the threaded holes 28, and pieces 1 and 2 are thereby joined forming a set 1-2 of the upper end of the implant.

Assembly of pieces 3 and 4: A piece 4 is selected which must be suitable for the angle of the lower vertebra V2 where it will rest on and it is put on top causing its anterior edges 31 and 41 to coincide with each other, on the one hand, and the rear edges 32 and 42 to coincide with each other, on the other hand. Four screws 10 are placed through the holes 46 and they are also threaded in the threaded holes 37, thereby forming a set 3-4 of the bottom end of the implant.

In this way, the two ends of implant I are obtained: on the one hand, set 1-2, and on the other hand, set 3-4, and when the ends 34 of the bars 35 are passed through the tubes 231, 232 and the tubes 23, the set 1-2 is fit by its end in set 3-4 by the other end so that the surgeon has in his side the extensions 15 of piece 1, and extension 45 of piece 4, as well as the four screws 20 of the telescopic fixation system. Two screws 20 are adjusted in the tubes 24, and the other two screws 20 are adjusted in tubes 26, which are oriented at an angle convergent on the space between the two tubes 24, in order to enable the adjustment by means of a screwdriver placed between both tubes 24.

3. Placement of the implant between two lumbar vertebrae: with implant I assembled in said way, and with the ends 34 of the bars 35 surpassing set 1-1, bars 35 are cut by their ends 34, two centimeters shorter than the spinal column defect, measuring the distance between the bottom edge of piece 4 and the ends 34 of bars 35. The implant is placed in the defect of the spinal column, thereby leaving set 3-4 resting on the lower vertebra V2 and the perpendicular bars 35 and between vertebrae V1 and V2 with their ends 34 under the upper vertebra V1, so that the anterior part of vertebrae V1 and V2 coincide with the anterior edges 11, 21, 31 and 41, and the rear part of the vertebral bodies V1 and V2 coincide with edges 12, 28, 32 and 42. In this way, the set 3-4 rests on vertebra V2 by the inside of its perimeter and with extension 45 resting on its outer face, and set 1-2 is in a middle point of bars 35, which ends face vertebra V1, thus being its projection within its perimeter. If necessary, in order to centralize sets 1-2 and 3-4 in the center of vertebrae V1 and V2, it may be necessary to make a small resection on the edges of both vertebrae, aiming to adapt the shape of the lateral extensions 14 and 44, as shown in FIG. 7B. Afterwards, separation of the ends formed by sets 1-2 and 3-4 is made manually or by means of separating instruments, and when freely slipping along the bars 35 the set 1-2, the separation of the platforms of the ends of implant I occurs, which turns from the position seen in FIG. 10A to the final position seen in FIG. 10B, the set 1-2 being applied by the inside of the upper vertebra V2 perimeter and its lateral extension 15 applied to the outer face. Screws 20 are placed and fixed in tubes 231, 232, and the two tubes 23, which fix the telescopic system, by using a screwdriver, oriented towards the screws 20 direction in order to block the telescopic system as shown in FIG. 9. The implant was thus placed straining its ends against the vertebrae. Screws 30 are placed through holes 18, and screws 301 are placed through holes 19 for piece 1, and vertebrae are fixed, for piece 4, screws 30 are placed through holes 47 and 301 for holes 48 and they are fixed to the vertebrae. In this way, the implant I mount to the vertebral defect between two lumbar vertebrae V1 and V2 is finished.

A variant in the coupling is used to be placed between a lumbar vertebra V1 from the top and the sacral bone S from the bottom. Owing to the different anatomical structures of the lumbar vertebra I and the sacral bone S, the sacral bone S requires a supporting piece 5, which is adjusted to piece 3 with screws 10 which penetrate into the holes 55 and are threaded and adjusted in holes 37. The mount is then carried out in another preferred embodiment with pieces 1, 2, 3 and 5 coupling, which for its accommodation in the spinal column defect between vertebrae V1 and S, has a mechanism identical to the above-described mechanism for the accommodation of implant I.

4. Once the implant has been placed, the whole space between the vertebrae is filled in with bone grafts and bioactive materials to form the bone callus which will include the implant thereby obtaining the definite mount fixation.

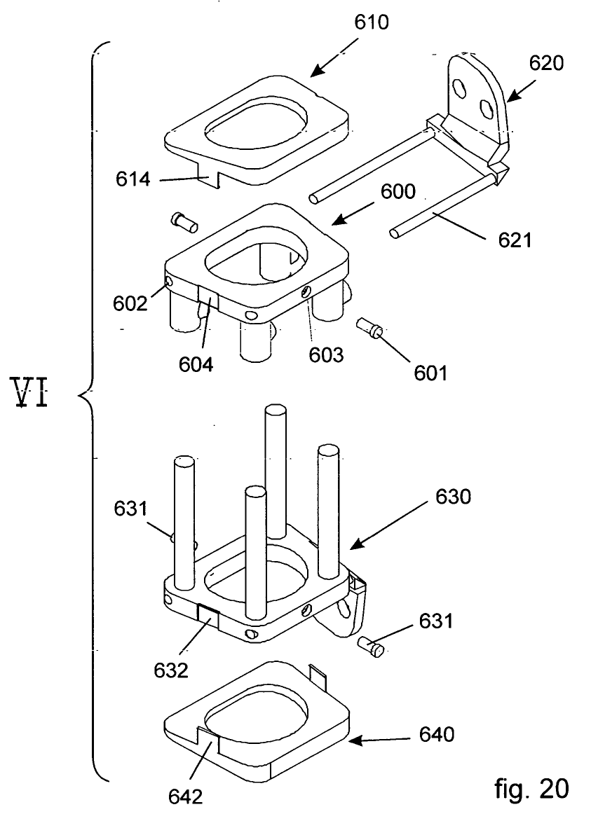

Another preferred embodiment of this invention, and with relation to the way the implant pieces couple, is illustrated in FIG. 20. For the 4-piece implant, the upper vertebral supporting piece is divided into two subpieces: one angular correction subpiece 610, which is adjusted to the telescopic adjusting piece 600 by means of flat projections 614 which fit in lifting spaces 604. In the same way, the bottom vertebral supporting piece is divided into two subpieces of identical configuration. The second subpiece 620 consists of the vertical extension including two horizontal bars 621, which will be introduced in the holes 602 of the new telescopic adjusting piece 600. The subpiece 620 is fixed in the piece 600 by means of the bar adjustment with screws 601, which are introduced in the lateral holes 603 of the telescopic adjusting piece 600. A configuration of this nature enables the reduction of the number of manual adjustments to be made to make the implant, and as a consequence only the adjustment of 4 screws is required for the assembly of all the pieces.

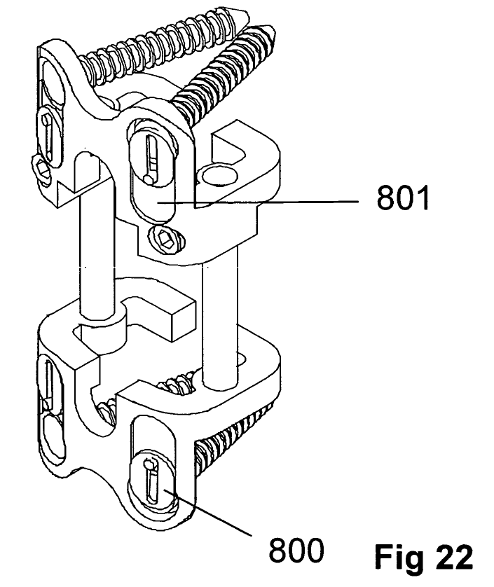

Another variant may be presented in the blocking system of the screws that are introduced in the body. This variant consists of slipping stops in order to prevent the fixed screw from a longitudinal displacement, as shown in FIG. 22. A grove is made on the lateral extension where said screws are, said groove having a rectangular shape with upper and bottom semicircular ends. One end of the groove will have the hole for the screw passage. The other end will have a circular shaped slipping stop and a groove to serve as a guide for the headed-bar type projection. Said guide-groove is vertically oriented in order to allow the screw head to slip and continues with an extension towards the circumference direction which allows it to rotate and thereby preventing the stop from a vertical slipping.

Another variant related to the orientation of the tube holes receiving the fixation screw 20 is illustrated in FIGS. 9A and 9B, wherein the longitudinal axis directions of the tube holes located at the longest distance from the surgeon's access converge on a point located in the middle of the side defined by the two tubes most accessible to the surgeon, so that said orientation enables the introduction of instruments for adjusting screws in the less accessible tubes.

FIG. 22 further illustrates a variant wherein the telescopic adjusting tube is not necessary by means of a hole in the horizontal member of the upper piece of the implant. Said variation is obtained by increasing the width of the horizontal member in the section of the original location of the tubes. The fixing screws are placed in holes made in the lateral faces of the horizontal member which height has been modified.

<newe text> After having described and invented the several preferred embodiments of this invention, which are described in the priority document, there followed the step of prototype making and tests in relation with the mechanical loads and practicity in the use of the device. A series of adaptations and revisions, which are described hereinbelow, has arisen from said tests.

After having verified the mechanical resistance of the structure and the materials used at present for this kind of device, it has been determined that the model for the lumbar area, the dorsal area and the cervical area will comprise two bars with their corresponding tubes. With regard to the parts forming the supports of the tubes, and similar to the proposal as an alternative embodiment for the cervical area device, it has been decided to use a top part instead of a supporting part and an adjusting part. FIG. A shows a two-bar device to be applied to the lumbar area. FIG. D shows the same device to be applied to the cervical area. In the same way, the thoracic area proceeds respecting the substantially triangular shape of said area vertebrae.

The top part (1010. FIG. A) is a vertebral supporting frame which shape is selected from the group comprised of a trapezoidal ring or a triangular ring, or a rectangular ring with a central protuberance in each short side representing two “E” shapes facing each other. From their short sides facing each other, tubes that will receive the bars emerge. Said tubes enable the bar slipping for the adjustment of device height. The bars will then be fixed to the tubes by means of fastening means, preferably screws entering through screwed holes located at the side walls of the tubes and in different spatial plans, as indicated in FIG. E (1060), which perpendicular direction must be free to enable the screws adjustment. The side opposite the vertebrae has a wedge-like shape and the oblique side of the wedge faces the vertebral plate. In this way, the insertion of the vertebral replacement into the corresponding place is enabled. The tubes of the top part are not located exactly in the side centers but are moved forward in order to make use, if necessary, of the remaining bone of the injured vertebra, thus favoring together with the bone grafts, cements and/or equivalent material, the fusion and consequent device fixing.

In view of the fact that the tubes are at a right-angle with the vertebral supporting frame, it is necessary to provide the part with angular correction ability. In order to obtain the bending of the top part, in order to restore the spinal curvature, a removable jig (1040 FIG. B) having the shape of the corresponding frame and a wedge-like shape which angles are predetermined. In order to prevent the jig from slipping with regard to the vertebral supporting frame, the jig has protuberances on its lower surface which fix in the corresponding incuts (1042) located on the inner merimeter of the ring of the vertebral supporting frame. It further has an incut (1043) on the outer perimeter of the part in order to enable the removal of the jig.

It becomes evident that now it is the jig the one leaning on the vertebral plate. In order to avoid any slipping between the jig and the vertebral plate, the jig comprises on its top surface a series of orientated wings offering resistance against slipping. The material recommended consists in titanium microgranules.

Furthermore, the top part and the bottom part may have inclinations as indicated in FIG. C (1011, 1012, 1013). The inclinations may be formed by any angle and parts of 0 degree, 5 degrees and 9.5 degrees are proposed. Said inclinations in combination with the jigs, which wedge-like shape may form angles of 0 degree, 1 degree, 2 degrees and 3 degrees, provide the set with a wide variety of inclinations suitable for the angular correction between the vertebrae.

Within the range of possible adaptations of this invention and as indicated in FIG. E, the part (1050) shows an orientated-wing type surface directly applied onto its surface instead of using the jig.

The vertebral supporting frame also has a vertical extension on the end of its short shides, in order to adjust the device to the vertebral face. Said vertical extension has a curved shape that is adapted to the lateral vertebral face. It also includes two screwed holes and orientated in different spatial plans in order to achieve a spatial fixing which may prevent the device from slipping.

With reference to the vertical extension, it is desirable that for some vertebrae there exists a horizontal slipping with regard to the frame original edge for its correct location and fixing as indicated in FIG. C (1090 and 1018). Said slippings are at predetermined distances.

With regard to the lower part (1020 FIG. A), which is the lower vertebral supporting frame, the same considerations as the ones for the top part may be applied, except that it lacks the tubes through which the bars slip (1035) and instead it has small threaded tubes wherein the bars are screwed. The frame shape, the fastening means, the slipping of the threaded tubes and of the vertical member are all in accordance with the top part. It is also possible to use the corresponding angular correction jig, which may have an inclination different from the inclination of the top part according to the desired correction. It is to be reminded that, according to the description in the priority document, there exist particular adaptations, both in the top part and the bottom part in relation to the end spinal vertebrae, that are valid for this preferred embodiment.

With regard to the bars (1035) that coil around the bottom part and get inserted into the tubes of the top part, they are substantially cylindrical and have a threaded bottom end and top end having a nut shape in order to facilitate its coiling around part B. The bars further have a mark which determines the extent they may slip within the receiving tube of part A. The bars have predetermined heights (FIG. C 1036/37/38/39) and have been designed to provide a height adjustment of 11 mm.

Another possibility for the present invention consists in placing a screen in the back area in order to avoid the migration of bone material or cement our of the intervertebral space. Therefore, the top part has a hole (1090) as indicated in FIG. E, which holds said screen.

With regard to the facing letter “E” (1070 FIG. E), characteristic of the adaptation to the cervical area of the present invention, the possibility of having the ends joined has been considered in order to provide the whole set with stability as indicated in FIG. E (1080). This modification does not enable the use of the proposed placing tools but enables the use of standard tools.

As it can be seen in this description, there exist variations that, in spite of being considered as technical equivalents in the original description, have practical improvements. Among said improvements, the redesign of the pieces for their assembly and the improvement in the interchangeability of bars to adjust the height of the vertebral replacement and the jig providing angular correction which is manually placed without the need of adjusting screws as was the case of the original description are to be mentioned. Likewise, the reduction to two bars for any region of the spinal column stands out since it improves the vision line and provides more space for the insertion of bone grafts thus favoring the device fixing.

What is claimed:

1. An implant for vertebral replacement comprised by inner supports to the vertebral plates, vertebral separating means and vertical members for adjusting the implant to the corresponding vertebral faces, wherein the piece of the inner support implant emulates the shape of the cortical tissue area of the vertebral plate thereby forming a frame and leaving the spongy tissue area of the vertebral plate free.

2. The implant of claim 1, wherein the shape of the inner support in a lumbar vertebra has a shape of a trapezoidal ring.

3. The implant of claim 1, wherein the shape of the inner support in a thoracic vertebra is triangulate ring-shaped.

4. The implant of claim 1, wherein the shape of the inner support in a cervical vertebra is rectangular ring-shaped with a central projection en each small side representing a two letter “E” shape said E facing each other.

5. The implant of claim 2, wherein the separating means are supported on and fixed to the inner support.

6. The implant of claim 5, wherein the vertical member and the inner support constitutes just one piece, having, thus a one piece superior member and a one piece inferior member plus separating means.

7. The implant of claim, wherein the vertebral separating means constitute a set of bars and a set of tubes, corresponding with each other, the outer diameters of the bars and the inner diameters of the tubes having a size enabling the adjusted slipping of the bars into the tubes and the fixation thereof at a height determined by fixing means.

8. The implant of claim 6, wherein the superior piece and the inferior piece allows the insertion of a removable jig, having a shape corresponding to the frame and a wedge-like shape which angles are predetermined; pieces includes incuts for purposes of insertion and removal of the jig.

9. The implant of claim 7, wherein the horizontal part of the superior and inferior pieces may be inclined with respect to the vertical members, to provide angular correction to the spine.

10. The implant of claim 7, wherein both the tubes and the bars are fixed in middle points in at least two of the sides forming the inner support.

11. The implant of claim 7, wherein both the tubes and the bars are fixed between the center and the posterior area points in just two opposite sides of the inner support.

12. The implant of claim 7, wherein the bar diameter is lower than 8 mm.

13. The implant of claim 10, wherein the bar diameter is of 4 mm.

14. The implant of claim 7, wherein the bars a regrooved in order to obtain diameter sections lower than the bar predetermined diameter to cut the bars.

15. The implant of claim 14, wherein the bar grooving is indented and the corresponding tube includes a device for obstructing the bar slipping into the tube towards a direction preferably corresponding to the approach of the vertebral separating pieces and the telescopic adjustment.

16. The implant of claim 7, wherein it is composed of four pieces:
an upper supporting piece having a horizontal member and a vertical extension for its adjustment to the lateral vertebral face by means of screws;
a telescopic adjusting piece having a flat member suitable for its adjustment to the upper supporting piece and at least two vertical extensions being tubes perpendicular to the horizontal member; a piece of vertebral separation composed of a flat member and a number equal to the telescopic adjusting piece of vertical extensions being bars perpendicular to the horizontal member which will be introduced into the tubes of the telescopic adjusting tubes; a bottom supporting piece having a horizontal member and a vertical extension for its adjustment to the vertebral lateral face by means of screws.

17. The implant of claim 16, wherein the horizontal members of the upper and bottom supporting pieces, and the horizontal members of the telescopic adjusting piece, and the vertebral separating piece have the shape selected from the group defined by the vertebral plate cortical areas of the lumbar vertebrate with a vertebral ring shape, ring-shaped cervical vertebrae formed by two letters “E” facing each other and thoracic vertebrae with a triangulate ring shape.

18. The implant of claim 16, wherein the bottom supporting piece is composed of a flat member and a flat extension which is adapted to the sacral bone, said flat member having an adaptable rectangular ring shape and adjustable to the vertebral separating piece.

19. The implant of claim 16, wherein the upper and bottom supporting pieces are a horizontal wedged member forming an angle for the restoration of the spinal curvature.

20. The implant of claim 16, wherein the union of pieces to each other is manually made with adjusting and fixing screws.

21. The implant of claim 7, wherein it is comprised by two pieces: an upper supporting and telescopic adjusting piece having a horizontal member and a vertical extension for its adjustment to the vertebral lateral face by means of screws, and at least two vertical extensions being tubes perpendicular to the horizontal member; a bottom supporting and vertebral separating piece having a flat member and a number equal to the telescopic adjusting piece of vertical extensions being bars perpendicular to the horizontal member, which will be introduced into the tubes of the telescopic adjusting piece, and a vertical extension for its adjustment to the vertebral lateral face by means of screws.

22. The implant of claim 21, wherein the horizontal members of said pieces have the shape selected from the group defined by the cortical areas of the vertebral plates of lumbar vertebrae having a vertebral ring shape, cervical vertebrae with a shape formed by two letters “E” facing each other, and triangulate ring shaped thoracic vertebrae.

23. The implant of claim 21, wherein the horizontal members of the pieces are comprised by a flat member and a flat extension which is adapted to the sacral bone, with a rectangular ring shape.

24. The implant of claim 21, wherein the piece horizontal members are wedged shaped forming an angle for the restoration of the spinal curvature.

25. The implant of claim 16, wherein the union between supporting pieces and vertebral separation and telescopic adjusting pieces have reciprocal fitting mechanisms.

26. The implant of claim 16, wherein the vertebral supporting surfaces of said pieces have surfaces which are not smooth.

27. The implant of claim 26, wherein the vertebral supporting pieces have wrinkled surfaces.

28. The implant of claim 26, wherein the vertebral supporting pieces have indented surfaces.

29. The implant of claim 16, wherein the lateral extensions adjusting against the lateral vertebral face enable the insertion of screw oblique to the longitudinal axis of the vertebrae.

30. The implant of claim 16, wherein the vertebral supporting piece and the vertebral separating piece are comprised by a unique piece having three flat members forming at least obtuse angles between them, and its bottom member includes vertical bars; its middle flat member includes holes for the passage of screws, thus turning this configuration suitable for its placement in the axis bone.

31. The implant of claim 16, wherein the vertical extensions of the vertebral supporting pieces include a stop system to prevent the screws that penetrate into the vertebral body from their longitudinal slipping.

32. The implant of claim 31, wherein said stop system is comprised by a groove in the lateral extension where there are holes for the screws to penetrate into the vertebral body, said groove having a rectangular shape with upper and lower semicircular ends; an end of the groove will have a hole for the passage of the screw; the other end will have a slipping stop with a circular shape and a groove which serves as a guide to the headed bar type projection; and said guide-groove is vertically orientated in order to enable the slipping towards the screw head and continues with an extension following the direction of the circumference which enables it to rotate, thus avoiding the vertical slipping of the stop.

33. The implant of claim 7, wherein it is composed of 6 pieces:
an upper supporting piece and a bottom supporting piece having a horizontal member and which are indistinctly fixed to a telescopic adjusting and vertebral separation piece, by means of vertical projecting sheets that fit in lateral grooves of the telescopic adjusting or vertebral separation piece; an upper vertical extension piece and a bottom adjusting piece for their adjustment to the vertebral lateral face by means of screws, which includes holes arranged for said purpose and which are adjusted to the telescopic adjusting or vertebral separation piece by means of two horizontal bars, which insert in the corresponding horizontal holes in the telescopic adjusting or vertebral separating piece, which final fixation is carried out by means of screws fit in holes located in the lateral faces of the telescopic adjusting and vertebral separating pieces;
a telescopic adjusting piece composed of a flat member suitable for the adjustment thereof to the upper supporting piece, which includes grooves in at least tow of its lateral faces to be fit in the vertebral supporting pieces, and at least two holes horizontally extend in order to contain two tubes of the vertical extension piece, and at least two vertical extensions being tubes perpendicular to the horizontal member; a vertebral separating piece composed of a flat member and a number equal to the telescopic adjusting piece, of vertical extensions being bars perpendicular to the horizontal member and said bars will be introduced into the tubes of the telescopic adjustment piece; said vertebral separating piece includes grooves in at least two of its lateral faces to be fit in vertebral supporting pieces, and at least two holes horizontally extending to contain two tubes of the vertical extension piece.

34. The implant of claim 7, wherein the bars and tubes are fixed with a leaning in relation to the pieces holding them, thereby providing the implant set with a curvature.

35. The implant of claim 7, wherein it includes incuts and discontinuities for the use of nippers with stops that enable the vertebral separation and the placement of the implant by means of said instrument.

36. A method for the placement of an implant of claim 35, wherein it comprises placing the implant in nippers having stops in the ends thereof; placing the nippers ends in the ends of the plates of two opposite vertebrae by exerting pressure on the nippers in order to separate the intervertebral bodies together with the separation of the implant pieces; slipping the implant inside the intervertebral space using the nippers arms and the incuts of the implant as a guide; reducing the pressure on the nippers and remove it through the discontinuities in the frames of the implant horizontal members; definitely adjusting the bars to the tubes.

37. The implant of claim 8, wherein the removable jig includes on its top surface a series of orientated wings offering resistance against slipping.

38. The implant of claim 8, wherein the wedge-like shape of the jig may form angles of 0 degrees, 1 degree, 2 degrees and 3 degrees.

39. The implant of claim 7, wherein both the tubes and the bars are fixed in the vertices of the inner supports.

40. The implant of claim 9, wherein the inclination of horizontal part of the superior and inferior pieces with respect to the vertical members, to provide angular correction to the spine is selected from the following values: 0 degrees, 5 degrees and 9.5 degrees.

41. The implant of claim 7, wherein the vertical extension has a curved shaped to adjust the device to the lateral vertebral face.

42. The implant of claim 7, wherein the vertical extension has an horizontal slipping at predetermined distances.

43. The implant of claim 7, wherein the superior piece includes a removable optional screen fixed by a screw to the piece to be located in the anterior area to avoid the migration of bone out of the vertebral area.

44. The implant of claim 7, wherein includes just two bars and two corresponding tubes for separating means.

45. The implant of claim 8, wherein the jig is made of titanium microgranules.

46. The implant of claim 7 wherein the bars and tubes allows an adjustment in height of 11 mm.
